SEQUENCE LISTING (28 sequences)

SEQ ID NO: 1 - protein, 932 aa, synthetic construct
SADAQSFLNRVCGVSAARLTPCGTGTSTDVVYRAFDIYNDKVAGFAKFLKTNCCRFQEKDEDDNLIDSYFVVKRHTFSNYQHEETIYNLLKDCPAVAKHDFFKFRIDGDMVPHISRQRLTKYTMADLVYALRHFDEGNCDTLKEILVTYNCCDDDYFNKKDWYDFVENPDILRVYANLGERVRQALLKTVQFCDAMRNAGIVGVLTLDNQDLNGNWYDFGDFIQTTPGSGVPVVDSYYSLLMPILTLTRALTAESHVDTDLTKPYIKWDLLKYDFTEERLKLFDRYFKYWDQTYHPNCVNCLDDRCILHCANFNVLFSTVFPPTSFGPLVRKIFVDGVPFVVSTGYHFRELGVVHNQDVNLHSSRLSFKELLVYAADPAMHAASGNLLLDKRTTCFSVAALTNNVAFQTVKPGNFNKDFYDFAVSKGFFKEGSSVELKHFFFAQDGNAAISDYDYYRYNLPTMCDIRQLLFVVEVVDKYFDCYDGGCINANQVIVNNLDKSAGFPFNKWGKARLYYDSMSYEDQDALFAYTKRNVIPTITQMNLKYAISAKNRARTVAGVSICSTMTNRQFHQKLLKSIAATRGATVVIGTSKFYGGWHNMLKTVYSDVENPHLMGWDYPKCDRAMPNMLRIMASLVLARKHTTCCSLSHRFYRLANECAQVLSEMVMCGGSLYVKPGGTSSGDATTAYANSVFNICQAVTANVNALLSTDGNKIADKYVRNLQHRLYECLYRNRDVDTDFVNEFYAYLRKHFSMMILSDDAVVCFNSTYASQGLVASIKNFKSVLYYQNNVFMSEAKCWTETDLTKGPHEFCSQHTMLVKQGDDYVYLPYPDPSRILGAGCFVDDIVKTDGTLMIERFVSLAIDAYPLTKHPNQEYADVFHLYLQYIRKLHDELTGHMLDMYSVMLTNDNTSRYWEPEFYEAMYTPHTVLQ

SEQ ID NO: 2 - protein, 431 aa, synthetic construct
MEGSKEPAVLNPKDPRLKTDFEEAIFSKYTGNKIMLMDEYMEEAVDHYVGCLEPLDISVDPIPLESAMYGMDGLEALDLTTSAGFPYLLQGKKKRDIFNRHTRDTTEMTKMLEKYGVDLPFVTFVKDELRSREKVEKGKSRLIEASSLNDSVAMRVAFGNLYATFHSNPGTATGSAVGCDPDIFWSKIPILLDGEIFAFDYTGYDASLSPVWFACLKKVLIKLGYTHQTSFIDYLCHSVHLYKDRKYIVNGGMPSGSSGTSIFNTMINNIIIRTLLIRVYKGIDLDQFKMIAYGDDVIASYPHKIDPGLLAEAGKHYGLVMTPADKGTSFVDTNWENVTFLKRYFRADDQYPFLIHPVMPMKEIHESIRWTKDPRNTQDHVRSLCYLAWHNGEEAYDEFCRKIRSVPVGRALTLPAYSSLRRKWLDSFLER

SEQ ID NO: 3 - protein, 639 aa, synthetic construct
EEDVNLGSGTRAVVSCAEAPNMKIIGNRIERIRSEHAETWFFDENHPYRTWAYHGSYEAPTQGSASSLINGVVRLLSKPWDVVTGVTGIAMTDTTPYGQQRVFKEKVDTRVPDPQEGTRQVMSMVSSWLWKELGKHKRPRVCTKEEFINKVRSNAALGAIFEEEKEWKTAVEAVNDPRFWALVDKEREHHLRGECQSCVYNMMGKREKKQGEFGKAKGSRAIWYMWLGARFLEFEALGFLNEDHWMGRENSGGGVEGLGLQRLGYVLEEMSRIPGGRMYADDTAGWDTRISRFDLENEALITNQMEKGHRALALAIIKYTYQNKVVKVLRPAEKGKTVMDIISRQDQRGSGQVVTYALNTFTNLVVQLIRNMEAEEVLEMQDLWLLRRSEKVTNWLQSNGWDRLKRMAVSGDDCVVKPIDDRFAHALRFLNDMGKVRKDTQEWKPSTGWDNWEEVPFCSHHFNKLHLKDGRSIVVPCRHQDELIGRARVSPGAGWSIRETACLAKSYAQMWQLLYFHRRDLRLMANAICSSVPVDWVPTGRTTWSIHGKGEWMTTEDMLVVWNRVWIEENDHMEDKTPVTKWTDIPYLGKREDLWCGSLIGHRPRTTWAENIKNTVNMVRRIIGDEEKYMDYLSTQVRY

SEQ ID NO: 4 - protein, 628 aa, synthetic construct
MDVIGERIKRIKEEHNSTWHYDDENPYKTWAYHGSYEVKATGSASSMINGVVKLLTKPWDVVPMVTQMAMTDTTPFGQQRVFKEKVDTRTPRPLPGTRKVMGITAEWLWRTLGRNKRPRLCTREEFTKKVRTNAAMGAVFTEENQWDSAKAAVEDEEFWKLVDRERELHKLGKCGSCVYNMMGKREKKLGEFGKAKGSRAIWYMWLGVRYLEFEALGFLNEDHWFSRENSYSGVEGEGLHKLGYILRDISKIPGGAMYADDTAGWDTRITEDDLHNEEKIIQQMDPEHRQLANAIFKLTYQNKVVKVQRPTPTGTVMDIISRKDQRGSGQVGTYGLNTFTNMEAQLVRQMEGEGVLTKADLENPHLLEKKITQWLETKGVERLKRMAISGDDCVVKPIDDRFANALLALNDMGKVRKDIPQWQPSKGWHDWQQVPFCSHHFHELIMKDGRKLVVPCRPQDELIGRARISQGAGWSLRETACLGKAYAQMWSLMYFHRRDLRLASNAICSAVPVHWVPTSRTTWSIHAHHQWMTTEDMLTVWNRVWIEENPWMEDKTPVTTWENVPYLGKREDQWCGSLIGLTSRATWAQNIPTAIQQVRSLIGNEEFLDYMPSMKRFRKEEESEGAIW

SEQ ID NO: 5 - protein, 589 aa, synthetic construct
PVNTLEEVHEEKCYPPKLDELKEQLLLKKLQESASTANRSRYQSRKVENMKATIIQRLKRGCKLYLMAETPKVPTYRTVYPAPVYSPPINVRLSNPESAVAACNEFLARNYPTVSSYQITDEYDAYLDMVDGSESCLDRATFNPSKLRSYPKQHAYHAPSIRSAVPSPFQNTLQNVLAAATKRNCNVTQMRELPTLDSAVFNVECFKKFACNREYWEEFAASPIRITTENLTTYVTKLKGPKAAALLARTHNLLPLQDVPMDRFTVDMKRDVKVTPGTKHTEERPKVQVIQAAEPLATAYLCGIHRELVRRLNAVLLPNVHTLFDMSAEDFDAIIAAHFKPGDAVLETDIASFDKSQDDSLALTALMLLEDLGVDHSLLDLIEAAFGEISSCHLPTGTRFKFGAMMKSGMFLTLFVNTLLNITIASRVLEDRLTKSACAAFIGDDNIIHGVVSDELMAARCATWMNMEVKIIDAVVSQKAPYFCGGFILHDTVTGTACRVADPLKRLFKLGKPLAAGDEQDEDRRRALADEVIRWQRTGLIDELEKAVYSRYEVQGISVAVMSMATFASSRSNFEKLRGPVITLYGGPK

SEQ ID NO: 6 - protein, 570 aa, synthetic construct
MSYTWTGALITPCAAEESKLPINALSNSLLRHHNMVYATTSRSAGLRQKKVTFDRLQVLDDHYRDVLKEMKAKASTVKAKLLSVEEACKLTPPHSAKSKFGYGAKDVRNLSSKAVNHIHSVWKDLLEDTVTPIDTTIMAKNEVFCVQPEKGGRKPARLIVFPDLGVRVCEKMALYDVVSTLPQVVMGSSYGFQYSPGQRVEFLVNTWKSKKNPMGFSYDTRCFDSTVTENDIRVEESIYQCCDLAPEARQAIKSLTERLYIGGPLTNSKGQNCGYRRCRASGVLTTSCGNTLTCYLKASAACRAAKLQDCTMLVNGDDLVVICESAGTQEDAASLRVFTEAMTRYSAPPGDPPQPEYDLELITSCSSNVSVAHDASGKRVYYLTRDPTTPLARAAWETARHTPVNSWLGNIIMYAPTLWARMILMTHFFSILLAQEQLEKALDCQIYGACYSIEPLDLPQIIERLHGLSAFSLHSYSPGEINRVASCLRKLGVPPLRVWRHRARSVRARLLSQGGRAATCGKYLFNWAVKTKLKLTPIPAASQLDLSGWFVAGYSGGDIYHSLSRARPRG

SEQ ID NO: 7 - RNA, 59 nt, synthetic construct
  source 1..59: mol_type = other RNA
gggcaagtgcggagaagatctctcgagaattttttttttttttttttttgcgcgcgcgc

SEQ ID NO: 8 - RNA, 40 nt, synthetic construct
  source 1..40: mol_type = other RNA
ctctcgagaattttttttttttttttttttgcgcgcgcgc

SEQ ID NO: 9 - RNA, 20 nt, synthetic construct
  source 1..20: mol_type = other RNA
ttctgcgtagaagccttttg

SEQ ID NO: 10 - RNA, 15 nt, synthetic construct
  source 1..15: mol_type = other RNA
gcaatgttgttcctt

SEQ ID NO: 11 - RNA, 17 nt, synthetic construct
  source 1..17: mol_type = other RNA
gaggaagttgtagcacg

SEQ ID NO: 12 - RNA, 16 nt, synthetic construct
  source 1..16: mol_type = other RNA
attgcagcattgttag

SEQ ID NO: 13 - RNA, 23 nt, synthetic construct
  source 1..23: mol_type = other RNA
caggattgcgggtgccaatgtga

SEQ ID NO: 14 - RNA, 25 nt, synthetic construct
  source 1..25: mol_type = other RNA
tcttttggtgtattcaaggctccct

SEQ ID NO: 15 - RNA, 25 nt, synthetic construct
  source 1..25: mol_type = other RNA
cagttgcaacccatatgatgccgtc

SEQ ID NO: 16 - RNA, 25 nt, synthetic construct
  source 1..25: mol_type = other RNA
tttgttagcaccatagggaagtcca

SEQ ID NO: 17 - RNA, 25 nt, synthetic construct
  source 1..25: mol_type = other RNA
gcttctggcccagttcctaggtagt

SEQ ID NO: 18 - RNA, 29 nt, synthetic construct
  source 1..29: mol_type = other RNA
agaaataccatcttggactgagatctttc

SEQ ID NO: 19 - RNA, 20 nt, synthetic construct
  source 1..20: mol_type = other RNA
ttctgcgtagaagccttttg

SEQ ID NO: 20 - RNA, 15 nt, synthetic construct
  source 1..15: mol_type = other RNA
gcaatgttgttcctt

SEQ ID NO: 21 - RNA, 17 nt, synthetic construct
  source 1..17: mol_type = other RNA
gaggaagttgtagcacg

SEQ ID NO: 22 - RNA, 16 nt, synthetic construct
  source 1..16: mol_type = other RNA
attgcagcattgttag

SEQ ID NO: 23 - RNA, 23 nt, synthetic construct
  source 1..23: mol_type = other RNA
caggattgcgggtgccaatgtga

SEQ ID NO: 24 - RNA, 25 nt, synthetic construct
  source 1..25: mol_type = other RNA
tcttttggtgtattcaaggctccct

SEQ ID NO: 25 - RNA, 25 nt, synthetic construct
  source 1..25: mol_type = other RNA
cagttgcaacccatatgatgccgtc

SEQ ID NO: 26 - RNA, 25 nt, synthetic construct
  source 1..25: mol_type = other RNA
tttgttagcaccatagggaagtcca

SEQ ID NO: 27 - RNA, 25 nt, synthetic construct
  source 1..25: mol_type = other RNA
gcttctggcccagttcctaggtagt

SEQ ID NO: 28 - RNA, 29 nt, synthetic construct
  source 1..29: mol_type = other RNA
agaaataccatcttggactgagatctttc

(19) United States
(12) Patent Application Publication
(10) Pub. No.: US 2024/0288428 A1
(43) Pub. Date: Aug. 29, 2024

(54) RNA POLYMERASE ASSAY

(71) Applicants: Qiyi Xie, San Diego, CA (US); Yong Jiang, San Diego, CA (US)

(72) Inventors: Qiyi Xie, San Diego, CA (US); Yong Jiang, San Diego, CA (US)

(21) Appl. No.: 18/506,484

(22) Filed: Nov. 10, 2023

Related U.S. Application Data
(60) Provisional application No. 63/383,458, filed on Nov. 11, 2022.

(51) Int. Cl. G01N 33/569; G01N 33/53

(52) U.S. Cl. CPC G01N 33/56983; G01N 33/5308; G01N 2333/165

(57) ABSTRACT

Provided herein is a method for determining the activity of an RNA polymerase, in particular, an RNA-dependent RNA polymerase (RDRP) of a coronavirus. Also provided herein is a kit for determining the activity of an RNA polymerase.

### CROSS REFERENCE TO RELATED APPLICATION

This application claims the benefit of U.S. Provisional Application No. 63/383,458, filed Nov. 11, 2022; the disclosure of which is incorporated herein by reference in its entirety.

### FIELD

Provided herein is a method for determining the activity of an RNA polymerase, in particular, an RNA-dependent RNA polymerase (RDRP) of a coronavirus. Also provided herein is a kit for determining the activity of an RNA polymerase.

### REFERENCE TO A SEQUENCE LISTING

The present specification is being filed with a Sequence Listing entitled 805A013US01_SEQLIST_ST26.XML of 29,352 bytes in size and created Nov. 10, 2023; the content of which is incorporated herein by reference in its entirety.

### BACKGROUND

The COVID-19 pandemic is caused by a severe acute respiratory syndrome coronavirus-2 (SARS-COV-2), a positive-sense single-stranded RNA virus. Zhang et al., Science 2020, 368, 409-12; Dai et al., Science 2020, 368, 1331-5; Hillen et al., Nature 2020, 584, 154-6; Pollard et al., Physiol. Genomics 2020, 52, 549-57. As of November 2022, COVID-19 has resulted in over six hundred million infections and almost seven million deaths. The COVID-19 pandemic poses as a major public health threat to the world. Pollard et al., Physiol. Genomics 2020, 52, 549-57.

Remdesivir is a nucleoside inhibitor that has been approved by the FDA for emergency use against COVID-19. Tian et al., Eur. J. Med. Chem. 2021, 213, 113201. Remdesivir targets the RNA-dependent RNA polymerase (RDRP) of SARS-COV-2, an essential enzyme for viral RNA replication and a promising drug target for COVID-19. Hillen et al., Nature 2020, 584, 154-6; Jiang et al., Biochem. Biophys. Res. Commun. 2021, 538, 47-53. However, remdesivir has a limited impact on COVID-19. WHO Solidarity Trial Consortium, Lancet 2022, 399, 1941-53. For example, remdesivir has no significant effect on COVID-19 patients on ventilation. Id. Therefore, there is a need for an RNA polymerase assay for the development of a RDRP inhibitor against SARS-COV-2.

### SUMMARY OF THE DISCLOSURE

Provided herein is a method for determining the activity of an RNA polymerase, comprising the steps of:
(a) contacting the RNA polymerase with a substrate mixture comprising nucleoside triphosphates, a labeled nucleoside triphosphate, and an oligonucleotide template to produce a labeled oligonucleotide;
(b) contacting the labeled oligonucleotide with a capture oligonucleotide to capture the labeled oligonucleotide, wherein the capture oligonucleotide is immobilized onto a surface of a solid phase; and
(c) detecting the labeled oligonucleotide captured by the capture oligonucleotide.

Also provided herein is a method for determining the activity of an RNA polymerase, comprising the steps of:
(a) contacting the RNA polymerase with a substrate mixture comprising nucleoside triphosphates, a labeled nucleoside triphosphate, an oligonucleotide primer, and an oligonucleotide template in a buffer solution to produce a labeled oligonucleotide;
(b) contacting the labeled oligonucleotide with a capture oligonucleotide to capture the labeled oligonucleotide, wherein the capture oligonucleotide is immobilized onto a surface of a solid phase; and;
(c) detecting the labeled oligonucleotide captured by the capture oligonucleotide.

Additionally provided herein is a method for determining the activity of an RNA polymerase, comprising:
(a) immobilizing a capture oligonucleotide onto a surface of a solid phase;
(b) contacting the RNA polymerase with a substrate mixture comprising nucleoside triphosphates, a labeled nucleoside triphosphate, and an oligonucleotide template to produce a labeled oligonucleotide;
(c) contacting the labeled oligonucleotide with a capture oligonucleotide to capture the labeled oligonucleotide; and
(d) detecting the labeled oligonucleotide captured by the capture oligonucleotide.

Furthermore, provided herein is a method for determining the activity of an RNA polymerase, comprising the steps of:
(a) immobilizing a capture oligonucleotide onto a surface of a solid phase;
(b) contacting the RNA polymerase with a substrate mixture comprising nucleoside triphosphates, a labeled nucleoside triphosphate, an oligonucleotide primer, and an oligonucleotide template to produce a labeled oligonucleotide;
(c) contacting the labeled oligonucleotide with a capture oligonucleotide to capture the labeled oligonucleotide; and
(d) detecting the labeled oligonucleotide captured by the capture oligonucleotide.

Provided herein is a kit for determining the activity of an RNA polymerase, comprising an RNA polymerase, nucleoside triphosphates, a labeled nucleoside triphosphate, an oligonucleotide template, and a capture oligonucleotide.

Provided herein is a kit for determining the activity of an RNA polymerase, comprising an RNA polymerase, nucleoside triphosphates, a labeled nucleoside triphosphate, an oligonucleotide primer, an oligonucleotide template, and a capture oligonucleotide.

Provided herein is a kit for determining the activity of an RNA polymerase, comprising an RNA polymerase, nucleoside triphosphates, a labeled nucleoside triphosphate, an oligonucleotide template, and a capture oligonucleotide coated onto a solid phase.

Provided herein is a kit for determining the activity of an RNA polymerase, comprising an RNA polymerase, nucleoside triphosphates, a labeled nucleoside triphosphate, an oligonucleotide primer, an oligonucleotide template, and a capture oligonucleotide coated onto a solid phase.

### BRIEF DESCRIPTION OF THE DRAWINGS

FIG. 1 shows the determination of the enzymatic activity of a SARS-COV-2 RDRP, wherein “Neg Ctrl” represents that the RNA polymerase assay was performed in the absence of the SARS-COV-2 RDRP; “Pos Ctrl” represents that the RNA polymerase assay was performed in the absence of the SARS-COV-2 RDRP with the addition of a synthetic biotinylated RNA; “RDRP” represents that the RNA polymerase assay was performed with the SARS-COV-2 RDRP (0.4 μM) and an RNA template (0.1 nM) for 30 min without 2 mM EDTA; and “RDRP+EDTA” represents that the RNA polymerase assay was performed with the SARS-COV-2 RDRP (0.4 μM) and an RNA template (0.1 nM) for 30 min with 2 mM EDTA.

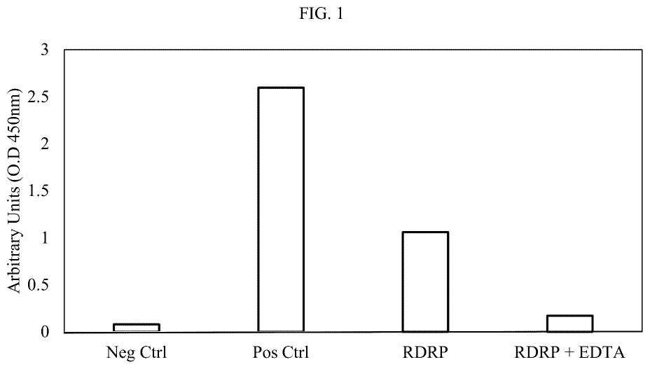

### DETAILED DESCRIPTION

To facilitate understanding of the disclosure set forth herein, a number of terms are defined below.

Generally, the nomenclature used herein and the laboratory procedures in biochemistry, biology, immunology, virology, and pharmacology described herein are those well-known and commonly employed in the art. Unless defined otherwise, all technical and scientific terms used herein generally have the same meaning as commonly understood by one of ordinary skill in the art to which this disclosure belongs.

The term “about” or “approximately” means an acceptable error for a particular value as determined by one of ordinary skill in the art, which depends in part on how the value is measured or determined. In certain embodiments, the term “about” or “approximately” means within 1, 2, or 3 standard deviations. In certain embodiments, the term “about” or “approximately” means within 25%, 20%, 15%, 10%, 9%, 8%, 7%, 6%, 5%, 4%, 3%, 2%, 1%, 0.5%, or 0.05% of a given value or range.

### RNA Polymerase Assay

In one embodiment, provided herein is a method of determining the activity of an RNA polymerase, comprising the steps of:
(a) contacting the RNA polymerase with a substrate mixture comprising nucleoside triphosphates, a labeled nucleoside triphosphate, and an oligonucleotide template to produce a labeled oligonucleotide;
(b) contacting the labeled oligonucleotide with a capture oligonucleotide to capture the labeled oligonucleotide, wherein the capture oligonucleotide is immobilized onto a surface of a solid phase; and
(c) detecting the labeled oligonucleotide captured by the capture oligonucleotide.

In another embodiment, provided herein is a method of determining the activity of an RNA polymerase, comprising the steps of:
(a) contacting the RNA polymerase with a substrate mixture comprising nucleoside triphosphates, a labeled nucleoside triphosphate, an oligonucleotide primer, and an oligonucleotide template in a buffer solution to produce a labeled oligonucleotide;
(b) contacting the labeled oligonucleotide with a capture oligonucleotide to capture the labeled oligonucleotide, wherein the capture oligonucleotide is immobilized onto a surface of a solid phase; and
(c) detecting the labeled oligonucleotide captured by the capture oligonucleotide.

In yet another embodiment, provided herein is a method of determining the activity of an RNA polymerase, comprising the steps of:
(a) immobilizing a capture oligonucleotide onto a surface of a solid phase;
(b) contacting the RNA polymerase with nucleoside triphosphates, a labeled nucleoside triphosphate, and an oligonucleotide template in a buffer solution to produce a labeled oligonucleotide;
(c) contacting the labeled oligonucleotide with the capture oligonucleotide to capture the labeled oligonucleotide; and
(d) detecting the labeled oligonucleotide captured by the capture oligonucleotide.

In still another embodiment, provided herein is a method of determining the activity of an RNA polymerase, comprising the steps of:
(a) coating a capture oligonucleotide onto a surface of a solid phase;
(b) contacting the RNA polymerase with nucleoside triphosphates, a labeled nucleoside triphosphate, an oligonucleotide primer, and an oligonucleotide template in a buffer solution to produce a labeled oligonucleotide;
(c) contacting the labeled oligonucleotide with a capture oligonucleotide to capture the labeled oligonucleotide; and
(d) detecting the labeled oligonucleotide captured by the capture oligonucleotide.

In certain embodiments, the labeled nucleoside triphosphate in a method provided herein is a radioactive nucleoside triphosphate. In certain embodiments, the labeled nucleoside triphosphate in a method provided herein is a ³H-, ¹⁴C-, ³²P-, ³³P-, ³⁵S-, or ¹²⁵I-nucleoside triphosphate.

In certain embodiments, the labeled nucleoside triphosphate in a method provided herein is a ³H-nucleoside triphosphate. In certain embodiments, the labeled nucleoside triphosphate in a method provided herein is a ³H-ATP. In certain embodiments, the labeled nucleoside triphosphate in a method provided herein is a ³H-CTP. In certain embodiments, the labeled nucleoside triphosphate in a method provided herein is a ³H-GTP. In certain embodiments, the labeled nucleoside triphosphate in a method provided herein is a ³H-UTP.

In certain embodiments, the labeled nucleoside triphosphate in a method provided herein is a ¹⁴C-nucleoside triphosphate. In certain embodiments, the labeled nucleoside triphosphate in a method provided herein is a ¹⁴C-ATP. In certain embodiments, the labeled nucleoside triphosphate in a method provided herein is a ¹⁴C-CTP. In certain embodiments, the labeled nucleoside triphosphate in a method provided herein is a ¹⁴C-GTP. In certain embodiments, the labeled nucleoside triphosphate in a method provided herein is a ¹⁴C-UTP.

In certain embodiments, the labeled nucleoside triphosphate in a method provided herein is a ³²P-nucleoside triphosphate. In certain embodiments, the labeled nucleoside triphosphate in a method provided herein is a ³²P-ATP. In certain embodiments, the labeled nucleoside triphosphate in a method provided herein is a ³²P-CTP. In certain embodiments, the labeled nucleoside triphosphate in a method provided herein is a ³²P-GTP. In certain embodiments, the labeled nucleoside triphosphate in a method provided herein is a ³²P-UTP.

In certain embodiments, the labeled nucleoside triphosphate in a method provided herein is a ³³P-nucleoside triphosphate. In certain embodiments, the labeled nucleoside triphosphate in a method provided herein is a ³³P-ATP. In certain embodiments, the labeled nucleoside triphosphate in a method provided herein is a ³³P-CTP. In certain embodiments, the labeled nucleoside triphosphate in a method provided herein is a ³³P-GTP. In certain embodiments, the labeled nucleoside triphosphate in a method provided herein is a ³³P-UTP.

In certain embodiments, the labeled nucleoside triphosphate in a method provided herein is an ³⁵S-nucleoside triphosphate. In certain embodiments, the labeled nucleoside triphosphate in a method provided herein is an ³⁵S-ATP. In certain embodiments, the labeled nucleoside triphosphate in a method provided herein is an ³⁵S-CTP. In certain embodiments, the labeled nucleoside triphosphate in a method provided herein is an ³⁵S-GTP. In certain embodiments, the labeled nucleoside triphosphate in a method provided herein is an ³⁵S-UTP.

In certain embodiments, the labeled nucleoside triphosphate in a method provided herein is an ¹²⁵I-nucleoside triphosphate. In certain embodiments, the labeled nucleoside triphosphate in a method provided herein is an ¹²⁵I-ATP. In certain embodiments, the labeled nucleoside triphosphate in a method provided herein is an ¹²⁵I-CTP. In certain embodiments, the labeled nucleoside triphosphate in a method provided herein is an ¹²⁵I-GTP. In certain embodiments, the labeled nucleoside triphosphate in a method provided herein is an ¹²⁵I-UTP.

In certain embodiments, the labeled nucleoside triphosphate in a method provided herein is a fluorescent nucleoside triphosphate. In certain embodiments, the labeled nucleoside triphosphate in a method provided herein is a nucleoside triphosphate labeled with a fluorescent dye. Suitable fluorescent dyes include, but are not limited to, an ATTO fluorescent dye (e.g., ATTO-488, ATTO-550, ATTO-590, ATTO-594, and ATTO-633), a cyanine dye (e.g., Cy2, Cy3, Cy3B, Cy3.5, Cy5, Cy5.5, and Cy7), a fluorescein dye (e.g., fluorescein isothiocyanate (FITC) and fluorescein diacetate), a rhodamine dye (e.g., rhodamine 6G, rhodamine 123, rhodamine B, rhodamine red, sulforhodamine B (SRB), sulforhodamine 101 (TEXAS RED™), carboxy-tetramethylrhodamine (TAMRA), tetramethylrhodamine (TMR), and tetramethylrhodamine isothiocyanate (TRITC)), an ALEXA FLUOR™ dye (e.g., ALEXA FLUOR™ 405, ALEXA FLUOR™ 530, ALEXA FLUOR™ 488, ALEXA FLUOR™ 500, ALEXA FLUOR™ 514, ALEXA FLUOR™ 532, ALEXA FLUOR™ 546, ALEXA FLUOR™ 555, ALEXA FLUOR™ 568, ALEXA FLUOR™ 594, ALEXA FLUOR™ 610, ALEXA FLUOR™ 633, ALEXA FLUOR™ 635, ALEXA FLUOR™ 647, ALEXA FLUOR™660, ALEXA FLUOR™680, ALEXA FLUOR™700, ALEXA FLUOR™750, and ALEXA FLUOR™ 790), a DYLIGHT™ FLUOR dye (e.g., DYLIGHT™ FLUOR 350, DYLIGHT™ FLUOR 405, DYLIGHT™ FLUOR 488, DYLIGHT™ FLUOR 550, DYLIGHT™ FLUOR 594, DYLIGHT™ FLUOR 633, DYLIGHT™ FLUOR 650, DYLIGHT™ FLUOR 680, DYLIGHT™ FLUOR 755, and DYLIGHT™ FLUOR 800), blue fluorescent protein (BFP), cyan fluorescent protein (CFP), green fluorescent protein (GFP), yellow fluorescent protein (YFP), and a phycobiliprotein (e.g., allophycoerythrin, B-phycoerythrin, C-phycoerythrin, or R-phycoerythrin).

In certain embodiments, the labeled nucleoside triphosphate in a method provided herein is a biotin-labeled nucleoside triphosphate, i.e., a biotinylated nucleoside triphosphate. In certain embodiments, the labeled nucleoside triphosphate in a method provided herein is a biotinylated ATP. In certain embodiments, the labeled nucleoside triphosphate in a method provided herein is a biotinylated CTP. In certain embodiments, the labeled nucleoside triphosphate in a method provided herein is a biotinylated GTP. In certain embodiments, the labeled nucleoside triphosphate in a method provided herein is a biotinylated UTP.

Thus, in one embodiment, provided herein is a method of determining the activity of an RNA polymerase, comprising the steps of:
(a) contacting the RNA polymerase with a substrate mixture comprising nucleoside triphosphates, a biotinylated nucleoside triphosphate, and an oligonucleotide template to produce a biotinylated oligonucleotide;
(b) contacting the biotinylated oligonucleotide with a capture oligonucleotide to capture the biotinylated oligonucleotide, wherein the capture oligonucleotide is immobilized onto a surface of a solid phase;
(c) contacting a biotin-binding protein with the biotinylated oligonucleotide captured by the capture oligonucleotide on the solid phase to capture the biotin-binding protein; and
(d) detecting the biotin-binding protein captured by the biotinylated oligonucleotide.

In another embodiment, provided herein is a method of determining the activity of an RNA polymerase, comprising the steps of:
(a) contacting the RNA polymerase with a substrate mixture comprising nucleoside triphosphates, a biotinylated nucleoside triphosphate, an oligonucleotide primer, and an oligonucleotide template in a buffer solution to produce a biotinylated oligonucleotide;
(b) contacting the biotinylated oligonucleotide with a capture oligonucleotide to capture the biotinylated oligonucleotide, wherein the capture oligonucleotide is immobilized onto a surface of a solid phase;
(c) contacting a biotin-binding protein with the biotinylated oligonucleotide captured by the capture oligonucleotide on the solid phase to capture the biotin-binding protein; and
(d) detecting the biotin-binding protein captured by the biotinylated oligonucleotide.

In yet another embodiment, provided herein is a method of determining the activity of an RNA polymerase, comprising the steps of:
(a) immobilizing a capture oligonucleotide onto a surface of a solid phase;
(b) contacting the RNA polymerase with nucleoside triphosphates, a biotinylated nucleoside triphosphate, and an oligonucleotide template in a buffer solution to produce a biotinylated oligonucleotide;
(c) contacting the biotinylated oligonucleotide with the capture oligonucleotide to capture the biotinylated oligonucleotide;
(d) contacting a biotin-binding protein with the biotinylated oligonucleotide captured by the capture oligonucleotide on the solid phase to capture the biotin-binding protein; and
(e) detecting the biotin-binding protein captured by the biotinylated oligonucleotide.

In still another embodiment, provided herein is a method of determining the activity of an RNA polymerase, comprising the steps of:
(a) immobilizing a capture oligonucleotide onto a surface of a solid phase;
(b) contacting the RNA polymerase with a substrate mixture comprising nucleoside triphosphates, a biotinylated nucleoside triphosphate, an oligonucleotide primer, and an oligonucleotide template in a buffer solution to produce a biotinylated oligonucleotide;
(c) contacting the biotinylated oligonucleotide with the capture oligonucleotide to capture the biotinylated oligonucleotide;
(d) contacting a biotin-binding protein with the biotinylated oligonucleotide captured by the capture oligonucleotide on the solid phase to capture the biotin-binding protein; and
(e) detecting the biotin-binding protein captured by the biotinylated oligonucleotide.

In certain embodiments, the biotin-binding protein is an avidin, streptavidin, or neutravidin. In certain embodiments, the biotin-binding protein is an avidin. In certain embodiments, the biotin-binding protein is a streptavidin. In certain embodiments, the biotin-binding protein is a neutravidin.

In certain embodiments, the biotin-binding protein is a labeled biotin-binding protein having a reporter. In certain embodiments, the biotin-binding protein is a labeled avidin, streptavidin, or neutravidin, each independently having a reporter. In certain embodiments, the biotin-binding protein is a labeled avidin having a reporter. In certain embodiments, the biotin-binding protein is a labeled streptavidin having a reporter. In certain embodiments, the biotin-binding protein is a labeled neutravidin having a reporter.

In certain embodiments, the reporter is a chromogenic reporter. In certain embodiments, the chromogenic reporter is an enzyme. In certain embodiments, the chromogenic reporter is a peroxidase. In certain embodiments, the chromogenic reporter is a horseradish peroxidase or alkaline peroxidase. In certain embodiments, the chromogenic reporter is a horseradish peroxidase. In certain embodiments, the chromogenic reporter is an alkaline peroxidase.

In certain embodiments, the biotin-binding protein described herein requires a substrate to generate a signal for detection. In certain embodiments, the substrate is a colorimetric substrate. In certain embodiments, the substrate is a fluorescent substrate. In certain embodiments, the substrate is a chemiluminescent substrate. In certain embodiments, the substrate is an electrochemiluminescent substrate. Suitable substrates for a horseradish peroxidase include, but are not limited to, 2,2′-azinobis(3-ethylbenzothiazoline-6-sulfonic acid) diammonium salt (ABTS), luminol, O-phenylenediamine dihydrochloride (OPD), 3,3′,5,5′-tetramethylbenzidine (TMB), AMPLEX™ RED, AMPLEX™ ULTRARED, QUANTABLU™, QUANTARED™, and SUPERSIGNAL™. Suitable substrates for an alkaline peroxidase include, but are not limited to, adamantyl 1,2-dioxetane aryl phosphate (AMPPD), p-nitrophenyl phosphate (PNPP), CDP-STAR™, CSPD™, and DYNALIGHT™.

In certain embodiments, the reporter is a colorimetric reporter. In certain embodiments, the reporter is a color particle. In certain embodiments, the reporter is a color microparticle or microsphere. In certain embodiments, the reporter is a color nanoparticle. In certain embodiments, the reporter is a gold particle. In certain embodiments, the reporter is a gold microparticle. In certain embodiments, the reporter is a gold nanoparticle. In certain embodiments, the reporter is a colloidal gold nanoparticle. In certain embodiments, the reporter is a colored latex particle. In certain embodiments, the reporter is a colored latex microparticle. In certain embodiments, the reporter is a colored latex nanoparticle. In certain embodiments, the reporter is a colored polystyrene particle. In certain embodiments, the reporter is a colored polystyrene microparticle. In certain embodiments, the reporter is a colored polystyrene nanoparticle.

In certain embodiments, the reporter is a fluorescent reporter. In certain embodiments, the reporter is an organic fluorophore. In certain embodiments, the reporter is a fluorescent protein. Suitable fluorescent reporters include, but are not limited to, a cyanine dye (e.g., Cy2, Cy3, Cy3B, Cy3.5, Cy5, Cy5.5, and Cy7), a fluorescein dye (e.g., fluorescein isothiocyanate (FITC) and fluorescein diacetate), a rhodamine dye (e.g., rhodamine 6G, rhodamine 123, rhodamine B, rhodamine red, sulforhodamine B (SRB), sulforhodamine 101 (TEXAS RED™), carboxytetramethylrhodamine (TAMRA), tetramethylrhodamine (TMR), and tetramethylrhodamine isothiocyanate (TRITC)), an ALEXA FLUOR™ dye (e.g., ALEXA FLUOR™ 405, ALEXA FLUOR™ 530, ALEXA FLUOR™ 488, ALEXA FLUOR™ 500, ALEXA FLUOR™ 514, ALEXA FLUOR™ 532, ALEXA FLUOR™ 546, ALEXA FLUOR™ 555, ALEXA FLUOR™ 568, ALEXA FLUOR™ 594, ALEXA FLUOR™ 610, ALEXA FLUOR™ 633, ALEXA FLUOR™ 635, ALEXA FLUOR™ 647, ALEXA FLUOR™660, ALEXA FLUOR™680, ALEXA FLUOR™700, ALEXA FLUOR™750, and ALEXA FLUOR™ 790), a DYLIGHT™ FLUOR dye (e.g., DYLIGHT™ FLUOR 350, DYLIGHT™ FLUOR 405, DYLIGHT™ FLUOR 488, DYLIGHT™ FLUOR 550, DYLIGHT™ FLUOR 594, DYLIGHT™ FLUOR 633, DYLIGHT™ FLUOR 650, DYLIGHT™ FLUOR 680, DYLIGHT™ FLUOR 755, and DYLIGHT™ FLUOR 800), blue fluorescent protein (BFP), cyan fluorescent protein (CFP), green fluorescent protein (GFP), yellow fluorescent protein (YFP), a phycobiliprotein (e.g., allophycoerythrin, B-phycoerythrin, C-phycoerythrin, or R-phycoerythrin), and a quantum dot.

In certain embodiments, the reporter is a chemiluminescent reporter. In certain embodiment, the chemiluminescent reporter is an acridinium or ruthenium ester. In certain embodiments, the reporter is an electrochemiluminescent reporter. In certain embodiments, the electrochemiluminescent reporter is tris(2,2′-bipyridyl)ruthenium (II) chloride or dichlorotris(1,10-phenanthroline)ruthenium (II).

In certain embodiments, the reporter is a radioactive reporter. In certain embodiments, the radioactive reporter is an ³H-, ¹⁴C-, ³²P-, ³³P-, ³⁵S-, or ¹²⁵I-reporter. In certain embodiments, the radioactive reporter is an ³H-reporter. In certain embodiments, the radioactive reporter is a ¹⁴C-reporter. In certain embodiments, the radioactive reporter is a ³²P-reporter. In certain embodiments, the radioactive reporter is a ³³P-reporter. In certain embodiments, the radioactive reporter is a ³⁵S-reporter. In certain embodiments, the radioactive reporter is a ¹²⁵I-reporter.

In certain embodiments, the biotin-binding protein is a labeled streptavidin having a chromogenic reporter. In certain embodiments, the biotin-binding protein is a labeled streptavidin conjugated with an enzyme as a reporter. In certain embodiments, the biotin-binding protein is a labeled streptavidin conjugated with a peroxidase as a reporter. In certain embodiments, the biotin-binding protein is a labeled streptavidin conjugated with a horseradish peroxidase as a reporter. In certain embodiments, the biotin-binding protein is a labeled streptavidin conjugated with a horseradish peroxidase as a reporter.

Thus, in one embodiment, provided herein is a method of determining the activity of an RNA polymerase, comprising the steps of:
(a) contacting the RNA polymerase with a substrate mixture comprising nucleoside triphosphates, a biotinylated nucleoside triphosphate, and an oligonucleotide template to produce a biotinylated oligonucleotide;
(b) contacting the biotinylated oligonucleotide with a capture oligonucleotide to capture the biotinylated oligonucleotide, wherein the capture oligonucleotide is immobilized onto a surface of a solid phase;
(c) contacting a peroxidase-streptavidin conjugate with the biotinylated oligonucleotide captured by the capture oligonucleotide on the solid phase to capture the peroxidase-streptavidin conjugate;
(d) contacting the peroxidase-streptavidin conjugate captured by the biotinylated oligonucleotide with a peroxidase substrate to generate a detectable signal; and
(e) detecting the detectable signal.

In another embodiment, provided herein is a method of determining the activity of an RNA polymerase, comprising the steps of:
(a) contacting the RNA polymerase with a substrate mixture comprising nucleoside triphosphates, a biotinylated nucleoside triphosphate, an oligonucleotide primer, and an oligonucleotide template in a buffer solution to produce a biotinylated oligonucleotide;
(b) contacting the biotinylated oligonucleotide with a capture oligonucleotide to capture the biotinylated oligonucleotide, wherein the capture oligonucleotide is immobilized onto a surface of a solid phase;
(c) contacting a peroxidase-streptavidin conjugate with the biotinylated oligonucleotide captured by the capture oligonucleotide on the solid phase to capture the peroxidase-streptavidin conjugate;
(d) contacting the peroxidase-streptavidin conjugate captured by the biotinylated oligonucleotide with a peroxidase substrate to generate a detectable signal; and
(e) detecting the detectable signal.

In yet another embodiment, provided herein is a method of determining the activity of an RNA polymerase, comprising the steps of:
(a) immobilizing a capture oligonucleotide onto a surface of a solid phase;
(b) contacting the RNA polymerase with nucleoside triphosphates, a biotinylated nucleoside triphosphate, and an oligonucleotide template in a buffer solution to produce a biotinylated oligonucleotide;
(c) contacting the biotinylated oligonucleotide with the capture oligonucleotide to capture the biotinylated oligonucleotide;
(d) contacting a peroxidase-streptavidin conjugate with the biotinylated oligonucleotide captured by the capture oligonucleotide on the solid phase to capture the peroxidase-streptavidin conjugate;
(e) contacting the peroxidase-streptavidin conjugate captured by the biotinylated oligonucleotide with a peroxidase substrate to generate a detectable signal; and
(f) detecting the detectable signal.

In still another embodiment, provided herein is a method of determining the activity of an RNA polymerase, comprising the steps of:
(a) immobilizing a capture oligonucleotide onto a surface of a solid phase;
(b) contacting the RNA polymerase with a substrate mixture comprising nucleoside triphosphates, a biotinylated nucleoside triphosphate, an oligonucleotide primer, and an oligonucleotide template in a buffer solution to produce a biotinylated oligonucleotide;
(c) contacting the biotinylated oligonucleotide with the capture oligonucleotide to capture the biotinylated oligonucleotide;
(d) contacting a peroxidase-streptavidin conjugate with the biotinylated oligonucleotide captured by the capture oligonucleotide on the solid phase to capture the peroxidase-streptavidin conjugate;
(e) contacting the peroxidase-streptavidin conjugate captured by the biotinylated oligonucleotide with a peroxidase substrate to generate a detectable signal; and
(f) detecting the detectable signal.

In certain embodiments, the peroxidase-streptavidin conjugate is a streptavidin conjugated with a horseradish peroxidase. In certain embodiments, the horseradish peroxidase substrate is a horseradish peroxidase substrate. In certain embodiments, the horseradish peroxidase substrate is 2,2′-azinobis(3-ethylbenzothiazoline-6-sulfonic acid) diammonium salt (ABTS), luminol, O-phenylenediamine dihydrochloride (OPD), 3,3′,5,5′-tetramethylbenzidine (TMB), AMPLEX™ RED, AMPLEX™ ULTRARED, QUANTABLU™, QUANTARED™, or SUPERSIGNAL™. In certain embodiments, the horseradish peroxidase substrate is 2,2′-azinobis(3-ethylbenzothiazoline-6-sulfonic acid) diammonium salt (ABTS). In certain embodiments, the horseradish peroxidase substrate is luminol. In certain embodiments, the horseradish peroxidase substrate is O-phenylenediamine dihydro-chloride (OPD). In certain embodiments, the horseradish peroxidase substrate is 3,3′,5,5′-tetramethyl-benzidine (TMB). In certain embodiments, the horseradish peroxidase substrate is AMPLEX™ RED. In certain embodiments, the horseradish peroxidase substrate is AMPLEX™ ULTRARED. In certain embodiments, the horseradish peroxidase substrate is QUANTABLU™. In certain embodiments, the horseradish peroxidase substrate is QUANTARED™. In certain embodiments, the horseradish peroxidase substrate is SUPERSIGNAL™.

In certain embodiments, the peroxidase-streptavidin conjugate is a streptavidin conjugated with an alkaline peroxidase. In certain embodiments, the peroxidase substrate is an alkaline peroxidase substrate. In certain embodiments, the alkaline peroxidase substrate is adamantyl 1,2-dioxetane aryl phosphate (AMPPD), p-nitrophenyl phosphate (PNPP), CDP-STAR™, CSPD™, or DYNALIGHT™. In certain embodiments, the alkaline peroxidase substrate is adamantyl 1,2-dioxetane aryl phosphate (AMPPD). In certain embodiments, the alkaline peroxidase substrate is p-nitrophenyl phosphate (PNPP). In certain embodiments, the alkaline peroxidase substrate is CDP-STAR™. In certain embodiments, the alkaline peroxidase substrate is CSPD™. In certain embodiments, the alkaline peroxidase substrate is DYNALIGHT™.

In one embodiment, the RNA polymerase is an RNA dependent RNA polymerase (RDRP). In another embodiment, the RNA polymerase is a viral RDRP. In yet another embodiment, the RNA polymerase is an RNA dependent RNA polymerase of a Chikungunya, a coronavirus, a dengue virus, an HCV, a poliovirus, or a Zika virus. In yet another embodiment, the RNA polymerase is an RDRP of a coronavirus. In yet another embodiment, the RNA polymerase is an RDRP of a SARS-COV-1, SARS-COV-2, or MERS-COV. In yet another embodiment, the RNA polymerase is an RDRP of a SARS-COV-1. In yet another embodiment, the RNA polymerase is an RDRP of a SARS-COV-2. In still another embodiment, the RNA polymerase is an RDRP of a MERS-COV.

In one embodiment, the RNA polymerase is recombinant. In another embodiment, the RNA polymerase is a recombinant RDRP. In yet another embodiment, the RNA polymerase is a recombinant RDRP of a coronavirus. In yet another embodiment, the RNA polymerase is a recombinant RDRP of SARS-COV-1, SARS-COV-2, or MERS-COV. In yet another embodiment, the RNA polymerase is a recombinant RDRP of a SARS-COV-1. In yet another embodiment, the RNA polymerase is a recombinant RDRP of a SARS-COV-2. In still another embodiment, the RNA polymerase is a recombinant RDRP of a MERS-COV.

In certain embodiments, the RNA polymerase is an RDRP comprising the amino acid sequence of SEQ ID NO: 1. In certain embodiments, the RNA polymerase is an RDRP comprising no less than about 80%, no less than about 85%, no less than about 90%, no less than about 91%, no less than about 92%, no less than about 93%, no less than about 94%, no less than about 95%, no less than about 96%, no less than about 97%, no less than about 98%, or no less than about 99% identical to the amino acid sequence of SEQ ID NO: 1. In certain embodiments, the RNA polymerase is an RDRP comprising no less than about 80% identical to the amino acid sequence of SEQ ID NO: 1. In certain embodiments, the RNA polymerase is an RDRP comprising no less than about 85% identical to the amino acid sequence of SEQ ID NO: 1. In certain embodiments, the RNA polymerase is an RDRP comprising no less than about 90% identical to the amino acid sequence of SEQ ID NO: 1. In certain embodiments, the RNA polymerase is an RDRP comprising no less than about 91% identical to the amino acid sequence of SEQ ID NO: 1. In certain embodiments, the RNA polymerase is an RDRP comprising no less than about 92% identical to the amino acid sequence of SEQ ID NO: 1. In certain embodiments, the RNA polymerase is an RDRP comprising no less than about 93% identical to the amino acid sequence of SEQ ID NO: 1. In certain embodiments, the RNA polymerase is an RDRP comprising no less than about 94% identical to the amino acid sequence of SEQ ID NO: 1. In certain embodiments, the RNA polymerase is an RDRP comprising no less than about 95% identical to the amino acid sequence of SEQ ID NO: 1. In certain embodiments, the RNA polymerase is an RDRP comprising no less than about 96% identical to the amino acid sequence of SEQ ID NO: 1. In certain embodiments, the RNA polymerase is an RDRP comprising no less than about 97% identical to the amino acid sequence of SEQ ID NO: 1. In certain embodiments, the RNA polymerase is an RDRP comprising no less than about 98% identical to the amino acid sequence of SEQ ID NO: 1. In certain embodiments, the RNA polymerase is an RDRP comprising no less than about 99% identical to the amino acid sequence of SEQ ID NO: 1.

In one embodiment, the RNA polymerase is a Chikungunya RDRP, a dengue virus RDRP, an HCV RDRP, a poliovirus RDRP, or a Zika virus RDRP. In another embodiment, the RNA polymerase is a Chikungunya RDRP. In yet another embodiment, the RNA polymerase is a dengue virus RDRP. In yet another embodiment, the RNA polymerase is an HCV, RDRP. In yet another embodiment, the RNA polymerase is a poliovirus RDRP. In still another embodiment, the RNA polymerase is a Zika virus RDRP.

In one embodiment, the RNA polymerase is a recombinant Chikungunya RDRP. In another embodiment, the RNA polymerase is a recombinant dengue virus RDRP. In yet another embodiment, the RNA polymerase is a recombinant HCV, RDRP. In yet another embodiment, the RNA polymerase is a recombinant poliovirus RDRP. In still another embodiment, the RNA polymerase is a recombinant Zika virus RDRP.

In certain embodiments, the RNA polymerase is an RDRP comprising the amino acid sequence of SEQ ID NO: 2. In certain embodiments, the RNA polymerase is an RDRP comprising no less than about 80%, no less than about 85%, no less than about 90%, no less than about 91%, no less than about 92%, no less than about 93%, no less than about 94%, no less than about 95%, no less than about 96%, no less than about 97%, no less than about 98%, or no less than about 99% identical to the amino acid sequence of SEQ ID NO: 2. In certain embodiments, the RNA polymerase is an RDRP comprising no less than about 80% identical to the amino acid sequence of SEQ ID NO: 2. In certain embodiments, the RNA polymerase is an RDRP comprising no less than about 85% identical to the amino acid sequence of SEQ ID NO: 2. In certain embodiments, the RNA polymerase is an RDRP comprising no less than about 90% identical to the amino acid sequence of SEQ ID NO: 2. In certain embodiments, the RNA polymerase is an RDRP comprising no less than about 91% identical to the amino acid sequence of SEQ ID NO: 2. In certain embodiments, the RNA polymerase is an RDRP comprising no less than about 92% identical to the amino acid sequence of SEQ ID NO: 2. In certain embodiments, the RNA polymerase is an RDRP comprising no less than about 93% identical to the amino acid sequence of SEQ ID NO: 2. In certain embodiments, the RNA polymerase is an RDRP comprising no less than about 94% identical to the amino acid sequence of SEQ ID NO: 2. In certain embodiments, the RNA polymerase is an RDRP comprising no less than about 95% identical to the amino acid sequence of SEQ ID NO: 2. In certain embodiments, the RNA polymerase is an RDRP comprising no less than about 96% identical to the amino acid sequence of SEQ ID NO: 2. In certain embodiments, the RNA polymerase is an RDRP comprising no less than about 97% identical to the amino acid sequence of SEQ ID NO: 2. In certain embodiments, the RNA polymerase is an RDRP comprising no less than about 98% identical to the amino acid sequence of SEQ ID NO: 2. In certain embodiments, the RNA polymerase is an RDRP comprising no less than about 99% identical to the amino acid sequence of SEQ ID NO: 2.

In certain embodiments, the RNA polymerase is an RDRP comprising the amino acid sequence of SEQ ID NO: 3. In certain embodiments, the RNA polymerase is an RDRP comprising no less than about 80%, no less than about 85%, no less than about 90%, no less than about 91%, no less than about 92%, no less than about 93%, no less than about 94%, no less than about 95%, no less than about 96%, no less than about 97%, no less than about 98%, or no less than about 99% identical to the amino acid sequence of SEQ ID NO: 3. In certain embodiments, the RNA polymerase is an RDRP comprising no less than about 80% identical to the amino acid sequence of SEQ ID NO: 3. In certain embodiments, the RNA polymerase is an RDRP comprising no less than about 85% identical to the amino acid sequence of SEQ ID NO: 3. In certain embodiments, the RNA polymerase is an RDRP comprising no less than about 90% identical to the amino acid sequence of SEQ ID NO: 3. In certain embodiments, the RNA polymerase is an RDRP comprising no less than about 91% identical to the amino acid sequence of SEQ ID NO: 3. In certain embodiments, the RNA polymerase is an RDRP comprising no less than about 92% identical to the amino acid sequence of SEQ ID NO: 3. In certain embodiments, the RNA polymerase is an RDRP comprising no less than about 93% identical to the amino acid sequence of SEQ ID NO: 3. In certain embodiments, the RNA polymerase is an RDRP comprising no less than about 94% identical to the amino acid sequence of SEQ ID NO: 3. In certain embodiments, the RNA polymerase is an RDRP comprising no less than about 95% identical to the amino acid sequence of SEQ ID NO: 3. In certain embodiments, the RNA polymerase is an RDRP comprising no less than about 96% identical to the amino acid sequence of SEQ ID NO: 3. In certain embodiments, the RNA polymerase is an RDRP comprising no less than about 97% identical to the amino acid sequence of SEQ ID NO: 3. In certain embodiments, the RNA polymerase is an RDRP comprising no less than about 98% identical to the amino acid sequence of SEQ ID NO: 3. In certain embodiments, the RNA polymerase is an RDRP comprising no less than about 99% identical to the amino acid sequence of SEQ ID NO: 3.

In certain embodiments, the RNA polymerase is an RDRP comprising the amino acid sequence of SEQ ID NO: 4. In certain embodiments, the RNA polymerase is an RDRP comprising no less than about 80%, no less than about 85%, no less than about 90%, no less than about 91%, no less than about 92%, no less than about 93%, no less than about 94%, no less than about 95%, no less than about 96%, no less than about 97%, no less than about 98%, or no less than about 99% identical to the amino acid sequence of SEQ ID NO: 4. In certain embodiments, the RNA polymerase is an RDRP comprising no less than about 80% identical to the amino acid sequence of SEQ ID NO: 4. In certain embodiments, the RNA polymerase is an RDRP comprising no less than about 85% identical to the amino acid sequence of SEQ ID NO: 4. In certain embodiments, the RNA polymerase is an RDRP comprising no less than about 90% identical to the amino acid sequence of SEQ ID NO: 4. In certain embodiments, the RNA polymerase is an RDRP comprising no less than about 91% identical to the amino acid sequence of SEQ ID NO: 4. In certain embodiments, the RNA polymerase is an RDRP comprising no less than about 92% identical to the amino acid sequence of SEQ ID NO: 4. In certain embodiments, the RNA polymerase is an RDRP comprising no less than about 93% identical to the amino acid sequence of SEQ ID NO: 4. In certain embodiments, the RNA polymerase is an RDRP comprising no less than about 94% identical to the amino acid sequence of SEQ ID NO: 4. In certain embodiments, the RNA polymerase is an RDRP comprising no less than about 95% identical to the amino acid sequence of SEQ ID NO: 4. In certain embodiments, the RNA polymerase is an RDRP comprising no less than about 96% identical to the amino acid sequence of SEQ ID NO: 4. In certain embodiments, the RNA polymerase is an RDRP comprising no less than about 97% identical to the amino acid sequence of SEQ ID NO: 4. In certain embodiments, the RNA polymerase is an RDRP comprising no less than about 98% identical to the amino acid sequence of SEQ ID NO: 4. In certain embodiments, the RNA polymerase is an RDRP comprising no less than about 99% identical to the amino acid sequence of SEQ ID NO: 4.

In certain embodiments, the RNA polymerase is an RDRP comprising the amino acid sequence of SEQ ID NO: 5. In certain embodiments, the RNA polymerase is an RDRP comprising no less than about 80%, no less than about 85%, no less than about 90%, no less than about 91%, no less than about 92%, no less than about 93%, no less than about 94%, no less than about 95%, no less than about 96%, no less than about 97%, no less than about 98%, or no less than about 99% identical to the amino acid sequence of SEQ ID NO: 5. In certain embodiments, the RNA polymerase is an RDRP comprising no less than about 80% identical to the amino acid sequence of SEQ ID NO: 5. In certain embodiments, the RNA polymerase is an RDRP comprising no less than about 85% identical to the amino acid sequence of SEQ ID NO: 5. In certain embodiments, the RNA polymerase is an RDRP comprising no less than about 90% identical to the amino acid sequence of SEQ ID NO: 5. In certain embodiments, the RNA polymerase is an RDRP comprising no less than about 91% identical to the amino acid sequence of SEQ ID NO: 5. In certain embodiments, the RNA polymerase is an RDRP comprising no less than about 92% identical to the amino acid sequence of SEQ ID NO: 5. In certain embodiments, the RNA polymerase is an RDRP comprising no less than about 93% identical to the amino acid sequence of SEQ ID NO: 5. In certain embodiments, the RNA polymerase is an RDRP comprising no less than about 94% identical to the amino acid sequence of SEQ ID NO: 5. In certain embodiments, the RNA polymerase is an RDRP comprising no less than about 95% identical to the amino acid sequence of SEQ ID NO: 5. In certain embodiments, the RNA polymerase is an RDRP comprising no less than about 96% identical to the amino acid sequence of SEQ ID NO: 5. In certain embodiments, the RNA polymerase is an RDRP comprising no less than about 97% identical to the amino acid sequence of SEQ ID NO: 5. In certain embodiments, the RNA polymerase is an RDRP comprising no less than about 98% identical to the amino acid sequence of SEQ ID NO: 5. In certain embodiments, the RNA polymerase is an RDRP comprising no less than about 99% identical to the amino acid sequence of SEQ ID NO: 5.

In certain embodiments, the RNA polymerase is an RDRP comprising the amino acid sequence of SEQ ID NO: 6. In certain embodiments, the RNA polymerase is an RDRP comprising no less than about 80%, no less than about 85%, no less than about 90%, no less than about 91%, no less than about 92%, no less than about 93%, no less than about 94%, no less than about 95%, no less than about 96%, no less than about 97%, no less than about 98%, or no less than about 99% identical to the amino acid sequence of SEQ ID NO: 6. In certain embodiments, the RNA polymerase is an RDRP comprising no less than about 80% identical to the amino acid sequence of SEQ ID NO: 6. In certain embodiments, the RNA polymerase is an RDRP comprising no less than about 85% identical to the amino acid sequence of SEQ ID NO: 6. In certain embodiments, the RNA polymerase is an RDRP comprising no less than about 90% identical to the amino acid sequence of SEQ ID NO: 6. In certain embodiments, the RNA polymerase is an RDRP comprising no less than about 91% identical to the amino acid sequence of SEQ ID NO: 6. In certain embodiments, the RNA polymerase is an RDRP comprising no less than about 92% identical to the amino acid sequence of SEQ ID NO: 6. In certain embodiments, the RNA polymerase is an RDRP comprising no less than about 93% identical to the amino acid sequence of SEQ ID NO: 6. In certain embodiments, the RNA polymerase is an RDRP comprising no less than about 94% identical to the amino acid sequence of SEQ ID NO: 6. In certain embodiments, the RNA polymerase is an RDRP comprising no less than about 95% identical to the amino acid sequence of SEQ ID NO: 6. In certain embodiments, the RNA polymerase is an RDRP comprising no less than about 96% identical to the amino acid sequence of SEQ ID NO: 6. In certain embodiments, the RNA polymerase is an RDRP comprising no less than about 97% identical to the amino acid sequence of SEQ ID NO: 6. In certain embodiments, the RNA polymerase is an RDRP comprising no less than about 98% identical to the amino acid sequence of SEQ ID NO: 6. In certain embodiments, the RNA polymerase is an RDRP comprising no less than about 99% identical to the amino acid sequence of SEQ ID NO: 6.

In certain embodiments, the RNA polymerase is isolated or purified.

In certain embodiments, each nucleoside triphosphate is independently a natural nucleoside triphosphate. In certain embodiments, each nucleoside triphosphate is independently adenosine triphosphate (ATP), cytidine triphosphate (CTP), guanosine triphosphate (GTP), or uridine triphosphate (UTP).

In certain embodiments, the oligonucleotide primer is an oligoribonucleotide. In certain embodiments, the oligonucleotide primer is an oligoribonucleotide comprising from about 2 to about 100, from about 2 to about 50, from about 2 to about 25, from about 2 to about 15, or from about 5 to about 10 ribonucleotides. In certain embodiments, the oligonucleotide primer is an oligoribonucleotide comprising from about 2 to about 100 ribonucleotides. In certain embodiments, the oligonucleotide primer is an oligoribonucleotide comprising from about 2 to about 50 ribonucleotides. In certain embodiments, the oligonucleotide primer is an oligoribonucleotide comprising from about 2 to about 25 ribonucleotides. In certain embodiments, the oligonucleotide primer is an oligoribonucleotide comprising from about 2 to about 15 ribonucleotides. In certain embodiments, the oligonucleotide primer is an oligoribonucleotide comprising from about 5 to about 10 ribonucleotides. In certain embodiments, the oligonucleotide primer is an oligoribonucleotide comprising from about 2, about 3, about 4, about 5, about 6, about 7, about 8, about 9, or about 10 ribonucleotides. In certain embodiments, the oligonucleotide primer is an oligoribonucleotide comprising from about 4, about 5, about 6, about 7, about 8, about 9, or about 10 ribonucleotides.

In certain embodiments, the oligonucleotide primer comprises an oligonucleotide sequence that is complementary to the oligonucleotide template employed in a method provided herein. In certain embodiments, the oligonucleotide primer comprises an oligonucleotide sequence that is complementary to the capture oligonucleotide employed in a method provided herein.

In certain embodiments, the oligonucleotide template is an oligoribonucleotide. In certain embodiments, the oligonucleotide template is an oligoribonucleotide comprising from about 5 to about 500, from about 5 to about 250, from about 5 to about 100, from about 10 to about 50, or from about 10 to about 25 ribonucleotides. In certain embodiments, the oligonucleotide template is an oligoribonucleotide comprising from about 5 to about 500 ribonucleotides. In certain embodiments, the oligonucleotide template is an oligoribonucleotide comprising from about 5 to about 250 ribonucleotides. In certain embodiments, the oligonucleotide template is an oligoribonucleotide comprising from about 5 to about 100 ribonucleotides. In certain embodiments, the oligonucleotide template is an oligoribonucleotide comprising from about 10 to about 50 ribonucleotides. In certain embodiments, the oligonucleotide template is an oligoribonucleotide comprising from about 10 to about 25 ribonucleotides. In certain embodiments, the oligonucleotide template is an oligoribonucleotide comprising from about 10, about 15, about 20, about 25, about 30, about 35, about 40, about 45, or about 50 ribonucleotides.

In certain embodiments, the oligonucleotide template is a single-stranded oligoribonucleotide. In certain embodiments, the oligonucleotide template is a double-stranded oligoribonucleotide.

In certain embodiments, the oligonucleotide template is an oligoribonucleotide comprising the sequence of any one of SEQ ID NOS: 9 to 28. In certain embodiments, the oligonucleotide template is an oligoribonucleotide comprising the sequence of any one of SEQ ID NOS: 9 to 18. In certain embodiments, the oligonucleotide template is an oligoribonucleotide comprising the sequence of any one of SEQ ID NOS: 19 to 28.

In certain embodiments, the oligonucleotide template is a single-stranded oligoribonucleotide comprising the sequence of any one of SEQ ID NOS: 9 to 28. In certain embodiments, the oligonucleotide template is a single-stranded oligoribonucleotide comprising the sequence of any one of SEQ ID NOS: 9 to 18. In certain embodiments, the oligonucleotide template is a single-stranded oligoribonucleotide comprising the sequence of any one of SEQ ID NOS: 19 to 28.

In certain embodiments, the oligonucleotide template is a double-stranded oligoribonucleotide comprising the sequence of any one of SEQ ID NOS: 9 to 28. In certain embodiments, the oligonucleotide template is a double-stranded oligoribonucleotide comprising the sequence of any one of SEQ ID NOS: 9 to 18. In certain embodiments, the oligonucleotide template is a double-stranded oligoribonucleotide comprising the sequence of any one of SEQ ID NOS: 19 to 28.

In certain embodiments, the oligonucleotide template comprises an oligonucleotide sequence that is complementary to the oligonucleotide primer employed in a method provided herein. In certain embodiments, the oligonucleotide template comprises an oligonucleotide sequence that is complementary to the labeled oligonucleotide generated by the RNA polymerase in a method provided herein.

In certain embodiments, the capture oligonucleotide is an oligoribonucleotide. In certain embodiments, the capture oligonucleotide is an oligoribonucleotide comprising from about 5 to about 500, from about 5 to about 250, from about 5 to about 100, from about 10 to about 50, or from about 10 to about 25 ribonucleotides. In certain embodiments, the capture oligonucleotide is an oligoribonucleotide comprising from about 5 to about 500 ribonucleotides. In certain embodiments, the capture oligonucleotide is an oligoribonucleotide comprising from about 5 to about 250 ribonucleotides. In certain embodiments, the capture oligonucleotide is an oligoribonucleotide comprising from about 5 to about 100 ribonucleotides. In certain embodiments, the capture oligonucleotide is an oligoribonucleotide comprising from about 10 to about 50 ribonucleotides. In certain embodiments, the capture oligonucleotide is an oligoribonucleotide comprising from about 10 to about 25 ribonucleotides. In certain embodiments, the capture oligonucleotide is an oligoribonucleotide comprising from about 10, about 15, about 20, about 25, about 30, about 35, about 40, about 45, or about 50 ribonucleotides.

In certain embodiments, the capture oligonucleotide is an oligoribonucleotide comprising the sequence of any one of SEQ ID NOS: 7 and 9 to 18.

In certain embodiments, the capture oligonucleotide is an oligonucleotide-protein conjugate, that is, a conjugate of an oligonucleotide and a protein. In certain embodiments, the protein in the capture oligonucleotide is an oligonucleotide-protein conjugate, wherein an alkaline phosphatase (AP), alpha-D-galactosidase (α-GAL), bovine serum albumin (BSA), C-reactive protein (CPR), glucose-6-phosphate dehydrogenase (G6PD), horseradish peroxidase (HRP), penicillinase (PNC), soybean peroxidase (SBP), or streptavidin. In certain embodiments, the capture oligonucleotide is an AP-oligonucleotide conjugate. In certain embodiments, the capture oligonucleotide is an α-GAL-oligonucleotide conjugate. In certain embodiments, the capture oligonucleotide is a CPR-oligonucleotide conjugate. In certain embodiments, the capture oligonucleotide is a G6PD-oligonucleotide conjugate. In certain embodiments, the capture oligonucleotide is an HRP-oligonucleotide conjugate. In certain embodiments, the capture oligonucleotide is a PNC-oligonucleotide conjugate. In certain embodiments, the capture oligonucleotide is an SBP-oligonucleotide conjugate. In certain embodiments, the capture oligonucleotide is a streptavidin-oligonucleotide conjugate.

In certain embodiments, the capture oligonucleotide comprises an oligonucleotide sequence that is complementary to the labeled oligonucleotide generated by the RNA polymerase in a method provided herein.

In certain embodiments, the capture oligonucleotide is immobilized covalently onto a surface of a solid phase. In certain embodiments, the capture oligonucleotide is immobilized noncovalently onto a surface of a solid phase.

In certain embodiments, the solid phase is a bead, a chip, a disc, a gel, a membrane, a sheet, a strip, or a well in a microplate.

In certain embodiments, the solid phase is a bead. In certain embodiments, the solid phase is a particle. In certain embodiments, the solid phase is a metal particle. In certain embodiments, the solid phase is a polymer bead. In certain embodiments, the solid phase is a microparticle. In certain embodiments, the solid phase is a nanoparticle. In certain embodiments, the solid phase is a microparticle, comprising copper, gold, platinum, or silver. In certain embodiments, the solid phase is a nanoparticle, comprising copper, gold, platinum, or silver. In certain embodiments, the solid phase is a carbon nanoparticle. In certain embodiments, the solid phase is a magnetic or paramagnetic bead. In certain embodiments, the solid phase is a bead comprising silica, latex, polyacrylate, polycarbonate, polyethylene, polyester, polypropylene, polystyrene, polyvinylidene difluoride (PVDF), or nylon. In certain embodiments, the solid phase is a latex bead. In certain embodiments, the solid phase is a polystyrene bead. In certain embodiments, the solid phase is a polycarbonate bead. In certain embodiments, the particle or bead comprises a reporter for detection.

In certain embodiments, the solid phase is a membrane. In certain embodiments, the solid phase is a porous membrane. In certain embodiments, the solid phase is a nitrocellulose, nylon, polyethersulfone, or PVDF membrane. In certain embodiments, the solid phase is a nitrocellulose membrane. In certain embodiments, the solid phase is a PVDF membrane. In certain embodiments, the solid phase is a sheet or strip. In certain embodiments, the solid phase is a nitrocellulose, nylon, polyethersulfone, or PVDF sheet. In certain embodiments, the solid phase is a nitrocellulose sheet. In certain embodiments, the solid phase is a PVDF sheet. In certain embodiments, the solid phase is a nitrocellulose, nylon, polyethersulfone, or PVDF strip. In certain embodiments, the solid phase is a nitrocellulose strip. In certain embodiments, the solid phase is a PVDF strip. In certain embodiments, the solid phase is a well in a microplate. In certain embodiments, the solid phase is a well in a polystyrene microplate. In certain embodiments, the solid phase is a microfluidic chip.

In certain embodiments, the solid phase is a biosensor. In certain embodiments, the solid phase is an SPR biosensor. In certain embodiments, the solid phase is a BLI biosensor.

In certain embodiments, the detecting step is performed colorimetrically. In certain embodiments, the detecting step is performed fluorescently. In certain embodiments, the detecting step is performed by chemiluminescence. In certain embodiments, the detecting step is performed by electrochemiluminescence. In certain embodiments, the detecting step is performed radioactively. In certain embodiments, the detecting step is performed using a biosensor. In certain embodiments, the detecting step is performed by surface plasmon resonance (SPR). In certain embodiments, the detecting step is performed by bio-layer interferometry (BLI).

In certain embodiments, the solid phase is not labeled.

### Kits

In one embodiment, provided herein is a kit for determining the activity of an RNA polymerase, comprising an RNA polymerase, nucleoside triphosphates, a labeled nucleoside triphosphate, an oligonucleotide template, and a capture oligonucleotide; wherein the RNA polymerase, nucleoside triphosphates, labeled nucleoside triphosphate, oligonucleotide template, and capture oligonucleotide are each as defined herein.

In another embodiment, provided herein is a kit for determining the activity of an RNA polymerase, comprising an RNA polymerase, nucleoside triphosphates, a labeled nucleoside triphosphate, an oligonucleotide primer, an oligonucleotide template, and a capture oligonucleotide; wherein the RNA polymerase, nucleoside triphosphates, labeled nucleoside triphosphate, oligonucleotide primer, oligonucleotide template, and capture oligonucleotide are each as defined herein.

In yet embodiment, provided herein is a kit for determining the activity of an RNA polymerase, comprising an RNA polymerase, nucleoside triphosphates, a labeled nucleoside triphosphate, an oligonucleotide template, and a capture oligonucleotide coated onto a solid phase; wherein the RNA polymerase, nucleoside triphosphates, labeled nucleoside triphosphate, oligonucleotide template, capture oligonucleotide, and solid phase are each as defined herein.

In still another embodiment, provided herein is a kit for determining the activity of an RNA polymerase, comprising an RNA polymerase, nucleoside triphosphates, a labeled nucleoside triphosphate, an oligonucleotide primer, an oligonucleotide template, and a capture oligonucleotide coated onto a solid phase; wherein the RNA polymerase, nucleoside triphosphates, labeled nucleoside triphosphate, oligonucleotide primer, oligonucleotide template, capture oligonucleotide, and solid phase are each as defined herein.

In one embodiment, provided herein is a kit for determining the activity of an RNA polymerase, comprising an RNA polymerase, nucleoside triphosphates, a biotinylated nucleoside triphosphate, an oligonucleotide template, a capture oligonucleotide, and a biotin-binding protein; wherein the RNA polymerase, nucleoside triphosphates, biotinylated nucleoside triphosphate, oligonucleotide template, capture oligonucleotide, and biotin-binding protein are each as defined herein.

In another embodiment, provided herein is a kit for determining the activity of an RNA polymerase, comprising an RNA polymerase, nucleoside triphosphates, a biotinylated nucleoside triphosphate, an oligonucleotide primer, an oligonucleotide template, a capture oligonucleotide, and a biotin-binding protein; wherein the RNA polymerase, nucleoside triphosphates, biotinylated nucleoside triphosphate, oligonucleotide primer, oligonucleotide template, capture oligonucleotide, and biotin-binding protein are each as defined herein.

In yet embodiment, provided herein is a kit for determining the activity of an RNA polymerase, comprising an RNA polymerase, nucleoside triphosphates, a biotinylated nucleoside triphosphate, an oligonucleotide template, a capture oligonucleotide coated onto a solid phase, and a biotin-binding protein; wherein the RNA polymerase, nucleoside triphosphates, biotinylated nucleoside triphosphate, oligonucleotide template, capture oligonucleotide, solid phase, and biotin-binding protein are each as defined herein.

In still another embodiment, provided herein is a kit for determining the activity of an RNA polymerase, comprising an RNA polymerase, nucleoside triphosphates, a labeled nucleoside triphosphate, an oligonucleotide primer, an oligonucleotide template, a capture oligonucleotide coated onto a solid phase, and a biotin-binding protein; wherein the RNA polymerase, nucleoside triphosphates, labeled nucleoside triphosphate, oligonucleotide primer, oligonucleotide template, capture oligonucleotide, solid phase, and biotin-binding protein are each as defined herein.

In one embodiment, provided herein is a kit for determining the activity of an RNA polymerase, comprising an RNA polymerase, nucleoside triphosphates, a biotinylated nucleoside triphosphate, an oligonucleotide template, a capture oligonucleotide, a peroxidase-streptavidin conjugate, and a peroxidase substrate; wherein the RNA polymerase, nucleoside triphosphates, biotinylated nucleoside triphosphate, oligonucleotide template, capture oligonucleotide, peroxidase-streptavidin conjugate, and peroxidase substrate are each as defined herein.

In another embodiment, provided herein is a kit for determining the activity of an RNA polymerase, comprising an RNA polymerase, nucleoside triphosphates, a biotinylated nucleoside triphosphate, an oligonucleotide primer, an oligonucleotide template, a capture oligonucleotide, a peroxidase-streptavidin conjugate, and a peroxidase substrate; wherein the RNA polymerase, nucleoside triphosphates, biotinylated nucleoside triphosphate, oligonucleotide primer, oligonucleotide template, capture oligonucleotide, peroxidase-streptavidin conjugate, and peroxidase substrate are each as defined herein.

In yet embodiment, provided herein is a kit for determining the activity of an RNA polymerase, comprising an RNA polymerase, nucleoside triphosphates, a biotinylated nucleoside triphosphate, an oligonucleotide template, a capture oligonucleotide coated onto a solid phase, a peroxidase-streptavidin conjugate, and a peroxidase substrate; wherein the RNA polymerase, nucleoside triphosphates, biotinylated nucleoside triphosphate, oligonucleotide template, capture oligonucleotide, solid phase, peroxidase-streptavidin conjugate, and peroxidase substrate are each as defined herein.

In still another embodiment, provided herein is a kit for determining the activity of an RNA polymerase, comprising an RNA polymerase, nucleoside triphosphates, a labeled nucleoside triphosphate, an oligonucleotide primer, an oligonucleotide template, a capture oligonucleotide coated onto a solid phase, a peroxidase-streptavidin conjugate, and a peroxidase substrate; wherein the RNA polymerase, nucleoside triphosphates, labeled nucleoside triphosphate, oligonucleotide primer, oligonucleotide template, capture oligonucleotide, solid phase, peroxidase-streptavidin conjugate, and peroxidase substrate are each as defined herein.

The disclosure will be further understood by the following non-limiting examples.

### EXAMPLES

### Example 1

### Recombinant RNA Dependent RNA Polymerase (RDRP)

A SARS-COV-2 RDRP gene encoding region was amplified by PCR from a SARS-COV-2 cDNA reverse-transcripted from a SARS-COV-2 viral RNA and cloned into AMERIDX® insect cell expression vector pADX50. After sequencing confirmation, the purified vector was transiently transfected into insect cell line Schneider 2 (S2). After induction with copper for seven days, the RDRP protein expressed was purified by an affinity Ni-NTA column and further purified by DETA SEPHAROSE chromatography.

### Example 2

### An RDRP Enzymatic Assay

A synthesized oligonucleotide of SEQ ID NO: 7 having an amino group at 5′-terminal was conjugated with a CRP protein using a MAGICLINK™ Oligo Antibody Conjugation Kit from BROADPHARM® to form a capture oligonucleotide. The capture oligonucleotide (100 ng/well) was added to each well in a 96-well plate. The plate was incubated for 4 hours at 37° C. and then blocked with Casein Blocking buffer (Sigma B6429) (Sigma B6429) for 1 hour at room temperature. Conjugated CRP-oligo complex was added in 96-well plate, 100 ng/well, incubate for 4 hours at 37° C., blocked with the Casein Blocking Buffer (Sigma) for 1 hour at room temperature.

An RDRP enzymatic reaction was carried out by incubating the SARS-COV-2 RDRP (0.4 μM) with an RNA template of SEQ ID NO: 8 (0.1 nM) for 30 min in the presence of ribonucleoside triphosphates (2.5 mM), about 25% by weight of which was a biotinylated UTP. The enzymatic reaction was carried out in a Tris-HCl buffer (500 mM, pH 8.5) containing 100 mM DTT, 25 mM MgCl₂, and 20 units RNasin at 30° C. for 1 hour. The enzymatic reaction was then stopped by adding a binding buffer (1×PBS with 0.1% TWEEN-20, 5 mM DTT, 20 mM sodium citrate, and 300 mM NaCl) (60 μL/well) and incubated at 70° C. for 5 minutes.

For detection, the enzymatic reaction mixture was added to a well of the 96-well plate coated with the capture oligonucleotide. After incubated at room temperature for 40 minutes on an orbit rotator, the 96-well plate was washed three times with an assay washing buffer (50 mM Tris-HCl, pH 8.0, 138 mM NaCl, 2.7 mM KCl, and 0.05% TWEEN-20). A streptavidin-HRP (1:10,000) was then added to each well of the 96-well plate. After incubated at room temperature for 40 minutes on an orbit rotator, the 96-well plate was washed three times with the assay washing buffer. An HRP substrate (SUREBLUE) was added to each well of the 96-well plate. The 96-well plate was included at room temperature for 5 to 15 minutes. A stop buffer (2M sulfuric acid) was added to each well of the 96-well plate. Absorbance at 450 nM was determined using an ELISA plate reader.

Sequences described herein are provided in the sequence table below.

SEQUENCE TABLE

| SEQ | | |
|---|---|---|
| ID | | |
| NO: | Description | Amino Acid or RNA Sequence |
| 1 | COVID-19 RDRP | SADAQSFLNRVCGVSAARLTPCGTG |
| | (Full Length) | TSTDVVYRAFDIYNDKVAGFAKFLK |
| | | TNCCRFQEKDEDDNLIDSYFVVKRH |
| | | TFSNYQHEETIYNLLKDCPAVAKHD |
| | | FFKFRIDGDMVPHISRQRLTKYTMA |
| | | DLVYALRHFDEGNCDTLKEILVTYN |
| | | CCDDDYFNKKDWYDFVENPDILRVY |
| | | ANLGERVRQALLKTVQFCDAMRNAG |
| | | IVGVLTLDNQDLNGNWYDFGDFIQT |
| | | TPGSGVPVVDSYYSLLMPILTLTRA |
| | | LTAESHVDTDLTKPYIKWDLLKYDF |
| | | TEERLKLFDRYFKYWDQTYHPNCVN |
| | | CLDDRCILHCANFNVLFSTVFPPTS |
| | | FGPLVRKIFVDGVPFVVSTGYHFRE |
| | | LGVVHNQDVNLHSSRLSFKELLVYA |
| | | ADPAMHAASGNLLLDKRTTCFSVAA |
| | | LTNNVAFQTVKPGNFNKDFYDFAVS |
| | | KGFFKEGSSVELKHFFFAQDGNAAI |
| | | SDYDYYRYNLPTMCDIRQLLFVVEV |
| | | VDKYFDCYDGGCINANQVIVNNLDK |
| | | SAGFPFNKWGKARLYYDSMSYEDQD |
| | | ALFAYTKRNVIPTITQMNLKYAISA |
| | | KNRARTVAGVSICSTMTNRQFHQKL |
| | | LKSIAATRGATVVIGTSKFYGGWHN |
| | | MLKTVYSDVENPHLMGWDYPKCDRA |
| | | MPNMLRIMASLVLARKHTTCCSLSH |
| | | RFYRLANECAQVLSEMVMCGGSLYV |
| | | KPGGTSSGDATTAYANSVFNICQAV |
| | | TANVNALLSTDGNKIADKYVRNLQH |
| | | RLYECLYRNRDVDTDFVNEFYAYLR |
| | | KHFSMMILSDDAVVCFNSTYASQGL |
| | | VASIKNFKSVLYYQNNVFMSEAKCW |
| | | TETDLTKGPHEFCSQHTMLVKQGDD |
| | | YVYLPYPDPSRILGAGCFVDDIVKT |
| | | DGTLMIERFVSLAIDAYPLTKHPNQ |
| | | EYADVFHLYLQYIRKLHDELTGHML |
| | | DMYSVMLTNDNTSRYWEPEFYEAMY |
| | | TPHTVLQ |
| 2 | Poliovirus RDRP | MEGSKEPAVLNPKDPRLKTDFEEAI |
| | (Full Length) | FSKYTGNKIMLMDEYMEEAVDHYVG |
| | | CLEPLDISVDPIPLESAMYGMDGLE |
| | | ALDLTTSAGFPYLLQGKKKRDIFNR |
| | | HTRDTTEMTKMLEKYGVDLPFVTFV |
| | | KDELRSREKVEKGKSRLIEASSLND |
| | | SVAMRVAFGNLYATFHSNPGTATGS |
| | | AVGCDPDIFWSKIPILLDGEIFAFD |
| | | YTGYDASLSPVWFACLKKVLIKLGY |
| | | THQTSFIDYLCHSVHLYKDRKYIVN |
| | | GGMPSGSSGTSIFNTMINNIIIRTL |
| | | LIRVYKGIDLDQFKMIAYGDDVIAS |
| | | YPHKIDPGLLAEAGKHYGLVMTPAD |
| | | KGTSFVDTNWENVTFLKRYFRADDQ |
| | | YPFLIHPVMPMKEIHESIRWTKDPR |
| | | NTQDHVRSLCYLAWHNGEEAYDEFC |
| | | RKIRSVPVGRALTLPAYSSLRRKWL |
| | | DSFLER |
| 3 | Zika Virus RDRP | EEDVNLGSGTRAVVSCAEAPNMKII |
| | | GNRIERIRSEHAETWFFDENHPYRT |
| | | WAYHGSYEAPTQGSASSLINGVVRL |
| | | LSKPWDVVTGVTGIAMTDTTPYGQQ |
| | | RVFKEKVDTRVPDPQEGTRQVMSMV |
| | | SSWLWKELGKHKRPRVCTKEEFINK |
| | | VRSNAALGAIFEEEKEWKTAVEAVN |
| | | DPRFWALVDKEREHHLRGECQSCVY |
| | | NMMGKREKKQGEFGKAKGSRAIWYM |
| | | WLGARFLEFEALGFLNEDHWMGREN |
| | | SGGGVEGLGLQRLGYVLEEMSRIPG |
| | | GRMYADDTAGWDTRISRFDLENEAL |
| | | ITNQMEKGHRALALAIIKYTYQNKV |
| | | VKVLRPAEKGKTVMDIISRQDQRGS |
| | | GQVVTYALNTFTNLVVQLIRNMEAE |
| | | EVLEMQDLWLLRRSEKVTNWLQSNG |
| | | WDRLKRMAVSGDDCVVKPIDDRFAH |
| | | ALRFLNDMGKVRKDTQEWKPSTGWD |
| | | NWEEVPFCSHHFNKLHLKDGRSIVV |
| | | PCRHQDELIGRARVSPGAGWSIRET |
| | | ACLAKSYAQMWQLLYFHRRDLRLMA |
| | | NAICSSVPVDWVPTGRTTWSIHGKG |
| | | EWMTTEDMLVVWNRVWIEENDHMED |
| | | KTPVTKWTDIPYLGKREDLWCGSLI |
| | | GHRPRTTWAENIKNTVNMVRRIIGD |
| | | EEKYMDYLSTQVRY |
| 4 | Dengue Virus | MDVIGERIKRIKEEHNSTWHYDDEN |
| | RDRP | PYKTWAYHGSYEVKATGSASSMING |
| | | VVKLLTKPWDVVPMVTQMAMTDTTP |
| | | FGQQRVFKEKVDTRTPRPLPGTRKV |
| | | MGITAEWLWRTLGRNKRPRLCTREE |
| | | FTKKVRTNAAMGAVFTEENQWDSAK |
| | | AAVEDEEFWKLVDRERELHKLGKCG |
| | | SCVYNMMGKREKKLGEFGKAKGSRA |
| | | IWYMWLGVRYLEFEALGFLNEDHWF |
| | | SRENSYSGVEGEGLHKLGYILRDIS |
| | | KIPGGAMYADDTAGWDTRITEDDLH |
| | | NEEKIIQQMDPEHRQLANAIFKLTY |
| | | QNKVVKVQRPTPTGTVMDIISRKDQ |
| | | RGSGQVGTYGLNTFTNMEAQLVRQM |
| | | EGEGVLTKADLENPHLLEKKITQWL |
| | | ETKGVERLKRMAISGDDCVVKPIDD |
| | | RFANALLALNDMGKVRKDIPQWQPS |
| | | KGWHDWQQVPFCSHHFHELIMKDGR |
| | | KLVVPCRPQDELIGRARISQGAGWS |
| | | LRETACLGKAYAQMWSLMYFHRRDL |
| | | RLASNAICSAVPVHWVPTSRTTWSI |
| | | HAHHQWMTTEDMLTVWNRVWIEENP |
| | | WMEDKTPVTTWENVPYLGKREDQWC |
| | | GSLIGLTSRATWAQNIPTAIQQVRS |
| | | LIGNEEFLDYMPSMKRFRKEEESEG |
| | | AIW |
| 5 | Chikungunya | PVNTLEEVHEEKCYPPKLDELKEQL |
| | RDRP | LLKKLQESASTANRSRYQSRKVENM |
| | | KATIIQRLKRGCKLYLMAETPKVPT |
| | | YRTVYPAPVYSPPINVRLSNPESAV |
| | | AACNEFLARNYPTVSSYQITDEYDA |
| | | YLDMVDGSESCLDRATFNPSKLRSY |
| | | PKQHAYHAPSIRSAVPSPFQNTLQN |
| | | VLAAATKRNCNVTQMRELPTLDSAV |
| | | FNVECFKKFACNREYWEEFAASPIR |
| | | ITTENLTTYVTKLKGPKAAALLART |
| | | HNLLPLQDVPMDRFTVDMKRDVKVT |
| | | PGTKHTEERPKVQVIQAAEPLATAY |
| | | LCGIHRELVRRLNAVLLPNVHTLFD |
| | | MSAEDFDAIIAAHFKPGDAVLETDI |
| | | ASFDKSQDDSLALTALMLLEDLGVD |
| | | HSLLDLIEAAFGEISSCHLPTGTRF |
| | | KFGAMMKSGMFLTLFVNTLLNITIA |
| | | SRVLEDRLTKSACAAFIGDDNIIHG |
| | | VVSDELMAARCATWMNMEVKIIDAV |
| | | VSQKAPYFCGGFILHDTVTGTACRV |
| | | ADPLKRLFKLGKPLAAGDEQDEDRR |
| | | RALADEVIRWQRTGLIDELEKAVYS |
| | | RYEVQGISVAVMSMATFASSRSNFE |
| | | KLRGPVITLYGGPK |
| 6 | HCV RDRP | MSYTWTGALITPCAAEESKLPINAL |
| | | SNSLLRHHNMVYATTSRSAGLRQKK |
| | | VTFDRLQVLDDHYRDVLKEMKAKAS |
| | | TVKAKLLSVEEACKLTPPHSAKSKF |
| | | GYGAKDVRNLSSKAVNHIHSVWKDL |
| | | LEDTVTPIDTTIMAKNEVFCVQPEK |
| | | GGRKPARLIVFPDLGVRVCEKMALY |
| | | DVVSTLPQVVMGSSYGFQYSPGQRV |
| | | EFLVNTWKSKKNPMGFSYDTRCFDS |
| | | TVTENDIR VEESIYQCCDLAPEAR |
| | | QAIKSLTERLYIGGPLTNSKGQNCG |
| | | YRRCRASGVLTTSCGNTLTCYLKAS |
| | | AACRAAKLQDCTMLVNGDDLVVICE |
| | | SAGTQEDAASLRVFTEAMTRYSAPP |
| | | GDPPQPEYDLELITSCSSNVSVAHD |
| | | ASGKRVYYLTRDPTTPLARAAWETA |
| | | RHTPVNSWLGNIIMYAPTLWARMIL |
| | | MTHFFSILLAQEQLEKALDCQIYGA |
| | | CYSIEPLDLPQHIERLHGLSAFSLH |
| | | SYSPGEINRVASCLRKLGVPPLRVW |
| | | RHRARSVRARLLSQGGRAATCGKYL |
| | | FNWAVKTKLKLTPIPAASQLDLSGW |
| | | FVAGYSGGDIYHSLSRARPRG |
| 7 | ON-1 | GGGCAAGUGCGGAGAAGAUCUCUCG |
| | | AGAAUUUUUUUUUUUUUUUUUUUUG |
| | | CGCGCGCGC |
| 8 | ON-2 | CUCUCGAGAAUUUUUUUUUUUUUUU |
| | | UUUUUGCGCGCGCGC |
| 9 | ON-3 | UUCUGCGUAGAAGCCUUUUG |
| 10 | ON-4 | GCAAUGUUGUUCCUU |
| 11 | ON-5 | GAGGAAGUUGUAGCACG |
| 12 | ON-6 | AUUGCAGCAUUGUUAG |
| 13 | ON-7 | CAGGAUUGCGGGUGCCAAUGUGA |
| 14 | ON-8 | UCUUUUGGUGUAUUCAAGGCUCCCU |
| 15 | ON-9 | CAGUUGCAACCCAUAUGAUGCCGUC |
| 16 | ON-10 | UUUGUUAGCACCAUAGGGAAGUCCA |
| 17 | ON-11 | GCUUCUGGCCCAGUUCCUAGGUAGU |
| 18 | ON-12 | AGAAAUACCAUCUUGGACUGAGAUC |
| | | UUUC |
| 19 | ON-13 | UUCUGCGUAGAAGCCUUUUG |
| 20 | ON-14 | GCAAUGUUGUUCCUU |
| 21 | ON-15 | GAGGAAGUUGUAGCACG |
| 22 | ON-16 | AUUGCAGCAUUGUUAG |
| 23 | ON-17 | CAGGAUUGCGGGUGCCAAUGUGA |
| 24 | ON-18 | UCUUUUGGUGUAUUCAAGGCUCCCU |
| 25 | ON-19 | CAGUUGCAACCCAUAUGAUGCCGUC |
| 26 | ON-20 | UUUGUUAGCACCAUAGGGAAGUCCA |
| 27 | ON-21 | GCUUCUGGCCCAGUUCCUAGGUAGU |
| 28 | ON-22 | AGAAAUACCAUCUUGGACUGAGAUC |
| | | UUUC |

The examples set forth above are provided to give those of ordinary skill in the art with a complete disclosure and description of how to make and use the claimed embodiments, and are not intended to limit the scope of what is disclosed herein. Modifications that are obvious to persons of skill in the art are intended to be within the scope of the following claims. All publications, patents, and patent applications cited in this specification are incorporated herein by reference as if each such publication, patent or patent application were specifically and individually indicated to be incorporated herein by reference.

1. A method for determining the activity of an RNA polymerase, comprising the steps of:
(a) contacting the RNA polymerase with a substrate mixture comprising nucleoside triphosphates, a labeled nucleoside triphosphate, and an oligonucleotide template to produce a labeled oligonucleotide;
(b) contacting the labeled oligonucleotide with a capture oligonucleotide to capture the labeled oligonucleotide, wherein the capture oligonucleotide is immobilized onto a surface of a solid phase; and
(c) detecting the labeled oligonucleotide captured by the capture oligonucleotide.

2. The method of claim 1, comprising the steps of:
(a) immobilizing a capture oligonucleotide onto a surface of a solid phase;
(b) contacting the RNA polymerase with a substrate mixture comprising nucleoside triphosphates, a labeled nucleoside triphosphate, and an oligonucleotide template to produce a labeled oligonucleotide;
(c) contacting the labeled oligonucleotide with the capture oligonucleotide to capture the labeled oligonucleotide; and
(d) detecting the labeled oligonucleotide captured by the capture oligonucleotide.

3. The method of claim 1, wherein the labeled nucleoside triphosphate is a radioactive nucleoside triphosphate, a fluorescent nucleoside triphosphate, or a biotin-labeled nucleoside triphosphate.

4-5. (canceled)

6. The method of claim 1, wherein the labeled nucleoside triphosphate is a biotinylated ATP, a biotinylated CTP, a biotinylated GTP, or a biotinylated UTP.

7. The method of claim 1, comprising the steps of:
(a) contacting the RNA polymerase with a substrate mixture comprising nucleoside triphosphates, a biotinylated nucleoside triphosphate, and an oligonucleotide template to produce a biotinylated oligonucleotide;
(b) contacting the biotinylated oligonucleotide with a capture oligonucleotide to capture the biotinylated oligonucleotide, wherein the capture oligonucleotide is immobilized onto a surface of a solid phase;
(c) contacting a biotin-binding protein with the biotinylated oligonucleotide captured by the capture oligonucleotide on the solid phase to capture the biotin-binding protein; and
(d) detecting the biotin-binding protein captured by the biotinylated oligonucleotide.

8. The method of claim 1, comprising the steps of:
(a) immobilizing a capture oligonucleotide onto a surface of a solid phase;
(b) contacting the RNA polymerase with a substrate mixture comprising nucleoside triphosphates, a biotinylated nucleoside triphosphate, and an oligonucleotide template to produce a labeled oligonucleotide;
(c) contacting the biotinylated oligonucleotide with the capture oligonucleotide to capture the biotinylated oligonucleotide;
(d) contacting a biotin-binding protein with the biotinylated oligonucleotide captured by the capture oligonucleotide on the solid phase to capture the biotin-binding protein; and
(e) detecting the biotin-binding protein captured by the biotinylated oligonucleotide.

9. The method of claim 7, wherein the biotin-binding protein is an avidin, streptavidin, or neutravidin.

10. The method of, wherein the biotin-binding protein is a streptavidin, a labeled streptavidin having a reporter, a labeled streptavidin having a chromogenic reporter, or a peroxidase-streptavidin conjugate.

11-13. (canceled)

14. The method of claim 1, comprising the steps of:
(a) contacting the RNA polymerase with a substrate mixture comprising nucleoside triphosphates, a biotinylated nucleoside triphosphate, and an oligonucleotide template to produce a biotinylated oligonucleotide;
(b) contacting the biotinylated oligonucleotide with a capture oligonucleotide to capture the biotinylated oligonucleotide, wherein the capture oligonucleotide is immobilized onto a surface of a solid phase;
(c) contacting a peroxidase-streptavidin conjugate with the biotinylated oligonucleotide captured by the capture oligonucleotide on the solid phase to capture the peroxidase-streptavidin conjugate;
(d) contacting the peroxidase-streptavidin conjugate captured by the biotinylated oligonucleotide with a peroxidase substrate to generate a detectable signal; and
(e) detecting the detectable signal.

15. The method of claim 1, comprising the steps of:
(a) immobilizing a capture oligonucleotide onto a surface of a solid phase;
(b) contacting the RNA polymerase with a substrate mixture comprising nucleoside triphosphates, a biotinylated nucleoside triphosphate, and an oligonucleotide template to produce a labeled oligonucleotide;
(c) contacting the biotinylated oligonucleotide with the capture oligonucleotide to capture the biotinylated oligonucleotide;
(d) contacting a peroxidase-streptavidin conjugate with the biotinylated oligonucleotide captured by the capture oligonucleotide on the solid phase to capture the peroxidase-streptavidin conjugate;
(e) contacting the peroxidase-streptavidin conjugate captured by the biotinylated oligonucleotide with a peroxidase substrate to generate a detectable signal; and
(f) detecting the detectable signal.

16. The method of claim 1, wherein the RNA polymerase is an RNA dependent RNA polymerase.

17-19. (canceled)

20. The method of claim 1, wherein the RNA polymerase is an RNA dependent RNA polymerase of a SARS-COV-2.

21-24. (canceled)

25. The method of claim 1, wherein the oligonucleotide template is an oligoribonucleotide of any one of SEQ ID NOS: 19 to 28.

26-27. (canceled)

28. The method of claim 1, wherein the capture oligonucleotide is an oligoribonucleotide-C-reactive protein (CPR) conjugate.

29. (canceled)

30. The method of claim 1, wherein the capture oligonucleotide comprises a sequence of any one of SEQ ID NOS: 7 and 9 to 18.

31. The method of claim 1, wherein the substrate mixture further comprises an oligonucleotide primer.

32. (canceled)

33. The method of claim 31, wherein the oligonucleotide primer is an oligoribonucleotide comprising from about 2 to about 100.

34. The method of any one of claims 1 to 33, wherein the solid phase is a bead, a chip, a disc, a gel, a membrane, a sheet, a strip, or a well in a microplate.

35-36. (canceled)

37. The method of claim 1, wherein the detecting step is performed colorimetrically.

38. A kit for determining the activity of an RNA polymerase, comprising an RNA polymerase, nucleoside triphosphates, a labeled nucleoside triphosphate, an oligonucleotide template, and a capture oligonucleotide.
